(19) United States
(12) Patent Application Publication
(10) Pub. No.: US 2011/0039354 A1
(43) Pub. Date: Feb. 17, 2011

(54) Method of Characterizing and Quantifying Calcifying Nanoparticles

(75) Inventors: James X. Hartman, Boca Raton, FL (US); Patricia Keating, Boca Raton, FL (US)

Correspondence Address: Gregory A. Nelson;Novak Druce & Quigg LLP, 525 Okeechobee Blvd, Suite 1500, West Palm Beach, FL, 33401, US

(73) Assignee: Florida Atlantic University

(21) Appl. No.: 12/934,354

(22) PCT Filed: Mar. 24, 2009
(86) PCT No.: PCT/US09/38066

§ 371 Date: Sep. 24, 2010

Related U.S. Application Data
(60) Provisional application No. 61/039,991, filed on Mar. 27, 2008.

(51) Int. Cl. G01N 33/53

(52) U.S. Cl. 436/501

(57) ABSTRACT

A method of characterizing calcifying nanoparticles (CNPs) can include creating a test sample comprising CNPs isolated from a biological source, a buffer solution, a plurality of calibration beads, and a fluorescent marker specifically linked to the CNPs; evaluating the test sample using a flow cytometer; and analyzing results from the flow cytometer to determine a characterizing feature of the calcifying nanoparticles. The characterizing feature of the calcifying nanoparticles can be the number of CNPs, concentration of CNPs, size of CNPs, level of CNP aggregation, size and light dispersion characteristics of CNPs, fluorescence intensity of the CNPs when labeled with a specific antibody, or a combinations thereof The method can also include evaluating an isotype control comprising CNPs isolated from the biological source, the buffer solution, a plurality of calibration beads, and a fluorescent marker that is not linked to the CNPs.

### TECHNICAL HELD

The invention relates to a method of characterizing and quantifying calcifying nanoparticles.

### BACKGROUND

Calcifying nanoparticles (CNPs), have been associated with a number of human diseases where the deposition of hydroxyapatite or other pathological forms of calcification in soft tissues, occurs. Calcifying nanoparticles (CNPs) have been identified in a wide variety of specimens, from soil to human serum and tissues. CNPs have been associated with calcifying diseases such as renal disease, rheumatoid arthritis, aortic aneurisms and calcific heart disease, prostatitis, psamomma bodies in ovarian cancer. The nature of CNPs is controversial but they most likely represent a non-living process of biomineralization around a protein nidi. Evidence supporting their characterization as replicating calcifying nanoparticles has been presented by a number of investigators. Ciftcioglu et al. have reported that the growth of CNPs can be inhibited by several antibiotics, primarily tetracyclines. Her group has shown that the optical density of a culture of a culture of the particles increases with incubation and aggregates appear to grow in number and size

Despite extensive research, CNPs have proven to be elusive to identify and characterize, mainly because of the shell of hydroxyapatite that surrounds them. The evidence linking these putative agents to human diseases has focused on the identification and characterization of CNPs by a number of methods including their filterability, enzyme linked immunoassay, electron microscopy and spectrophotometric analysis. Kajander has developed a monoclonal antibody, designated 8D10 that reacts specifically with CNPs from a variety of sources. The antibody was produced by demineralizing CNPs obtained from fetal bovine serum, subjecting them to extensive proteolysis with proteinase A, washing the CNPs, and injecting them into mice.

The 8D10 monoclonal antibody has been used by several groups to identify CNPs in human atherosclerotic plaque and aneurysms. Their western blot analysis showed that the antibody identifies a 50,000 Da protein extracted from CNPs. Kajander has used the 8D10 monoclonal antibody to identify CNPs by ELISA in bovine and human plasma and, after extensive demineralization, CNPs embedded within kidney stones.

A preliminary report has documented what appears to be the successful use of a calcium chelating agent and tetracycline in the therapy of prostatitis, presumably by reducing the number of CNPs. Tetracycline therapy is used in the prevention of recurring kidney stones by controlling the growth of CNPs. However, these investigators did not monitor the CNP load or the number of particles present following therapy, Enzyme linked immunoassays have been used to detect the presence of nanobacteria in tissue and serum samples but no quantitative data has been reported. Kajander et al. have stressed that there are limits to the current methodologies in the detection and cultivation of calcified nanoparticles.

### SUMMARY OF THE INVENTION

The present invention is directed to a method of characterizing and quantifying calcifying nanoparticles (CNPs). The method can include creating a test sample that contains CNPs isolated from a biological source, a buffer solution, a plurality of calibration beads, and a fluorescent marker specifically linked to the CNPs. The test sample can be evaluated using a flow cytometer and the results from the flow cytometer can be analyzed to determine a characterizing feature of the calcifying nanoparticles. The characterizing feature can be the number of CNPs, the concentration of CNPs, the size of CNPs, the level of CNP aggregation, the size and light dispersion characteristics of CNPs, the fluorescence intensity of the CNPs when labeled with a specific antibody, and combinations thereof.

The method can also include creating an isotype control that contains CNPs isolated from a biological source, the buffer solution, a plurality of calibration beads, and a fluorescent marker that is not linked to the CNPs. The isotype control can be evaluated using the flow cytometer. The test solution and the isotype control can have approximately the same concentration of beads and the beads can have a uniform diameter. The uniform diameter of the beads can be selected so that the beads are larger than the expected size of CNP aggregates in the test sample. The beads can have a uniform diameter ranging between 5 micrometers and 10 micrometers. The beads can be fluorescent.

The analysis of the flow cytometer results can include analyzing a plot of side scatter and forward scatter results from the flow cytometer evaluation of the test sample. The plot can be a log-log plot of side scatter and forward scatter results.

The selective linkage can include a monoclonal antibody that specifically binds to the CNPs. The selective linkage can include an antibody that specifically binds to the monoclonal antibody. The selective linkage can include a monoclonal antibody that specifically binds to the CNPs and another antibody that specifically binds to the monoclonal antibody.

The test sample can be produced by creating a first solution comprising CNPs isolated from a biological source, a buffer solution, and a monoclonal antibody that specifically binds to CNPs. The first sample can be incubated for a period of sufficient duration for the monoclonal antibody to bind to the CNPs in the first sample. Next, the test sample can be created by adding a marker to the first sample. The marker can include a fluorescent molecule and the marker can specifically bind to the monoclonal antibody. The CNPs isolated from the biological source can be obtained from blood, bodily exudates, abscess fluids, cells, tissue, extracted tissue, and combinations thereof.

The monoclonal antibody can be 8D10. The marker can include an antibody conjugated to a fluorescent molecule, where the antibody is produced against the monoclonal antibody.

Prior to creating the first solution, a liquid containing CNPs from the biological source can be filtered through a 0.2 micron filter. The filtrate from the filtering process can be used to create the test sample.

The isotype control can be produced by creating a control precursor comprising CNPs isolated from a mammalian subject, a buffer solution, and a monoclonal antibody that is non-specific for CNPs. The control precursor can be incubated for a period of approximately the same duration as the incubating step used to produce the test sample. The isotype control can then be created by adding the marker to the control precursor.

These and other embodiments are described in more detail below.

### BRIEF DESCRIPTION OF THE DRAWINGS

The invention is described with more particularity below. The above and further advantages of this invention may be better understood by referring to the following descriptions taken in conjunction with the accompanying figures, in which:

FIG. 1 is dot plots of microbeads dilutions determined by flow cytometry.

FIG. 2 is dot plots a demonstration that CNPs can be differentiated from 6 micron beads and their concentration approximated.

FIG. 3 is dot plots demonstrating that CNPs are filterable agents that can be detected and roughly sized by flow cytometry,

FIG. 4 is dot plots demonstrating that motile forms of CNPs can be differentiated from larger crystalline forms seen in older cultures.

FIG. 5 is dot plots demonstrating the effects of HCl on the distribution of CNPs in flow cytometric analysis.

FIGS. 6A & B are dot plots and FL1 histograms of non-gamma irradiated, non-40 nm filtered FBS samples analyzed by flow cytometry.

FIG. 7 is dot plots and FL1 histograms demonstrating the specificity of 8D10 versus irrelevant mouse monoclonal antibodies toward CNPs.

FIG. 8 is dot plots and FL1 histograms of FBS samples stained with either 8D10 monoclonal antibody or a mouse IgG negative control.

FIGS. 9A & 9B are three dimensional SSLog vs FL1 plots.

FIGS. 10A and 10B are dot plots and FL1 histograms demonstrating the effect of ultracentrifugation of FBS samples.

FIGS. 11A & 11B are dot plots and FL1 histograms analyzing CNPs from an SLE patient's plasma by flow cytometry using 8D10 monoclonal antibody.

FIG. 12 is a series of dot plots and FL1 histograms of three different samples of a healthy donor's uncultured plasma, using 8D10 monoclonal antibody.

FIG. 13 is a series of dot plots and FL1 histograms of an SLE patient's panniculitis exudate sample before, and 10 days after incubation in serum-free media.

FIG. 14 is a chart showing that the number of events, i.e., CNPs, increases with time of incubation.

FIG. 15 is a chart showing that the number of events, i.e., CNPs, increases on the FL-1 channel with time of incubation.

FIG. 16 is a chart showing that the number of events, i.e., CNPs, increases on FL-1 channel with time of incubation.

FIG. 17 is a FL1 histogram analyzing the shift toward lower mean fluorescence intensities (MFI) in the positive region for 8D10 monoclonal antibody.

FIG. 18 is a series of FL1 histograms showing the shift toward lower mean fluorescence intensity on FL-1.

FIGS. 19A and 19B are dot plots and FL1 histograms showing characteristics of size and light scattering properties of CNPs stained with 8D10 monoclonal antibody.

FIGS. 20A and 20B are plots showing characteristics of size and light scattering properties of CNPs stained with 8D10 monoclonal antibody.

### DETAILED DESCRIPTION

The present invention is directed to a method of characterizing and quantifying calcifying nanoparticles (CNPs). The method can include creating a test sample that contains CNPs isolated from a biological source, a buffer solution, a plurality of calibration heads, and a fluorescent marker specifically linked to the CNPs. The test sample can be evaluated using a flow cytometer and the results from the flow cytometer can be analyzed to determine a characterizing feature of the calcifying nanoparticles. The characterizing feature can be the number of CNPs, the concentration of CNPs, the size of CNPs, the level of CNP aggregation, the size and light dispersion characteristics of CNPs, the fluorescence intensity of the CNPs when labeled with a specific antibody, and combinations thereof.

The method can also include creating an isotype control that contains CNPs isolated from a biological source, the buffer solution, a plurality of calibration beads, and a fluorescent marker that is not linked to the CNPs. The isotype control can be evaluated using the flow cytometer. The test solution and the isotype control comprise approximately the same concentration of beads and the beads have a uniform diameter. The uniform diameter of the beads can be selected so that the beads are larger than the expected size of CNP aggregates in the test sample. The beads can have a uniform diameter ranging between 5 micrometers and 10 micrometers. The beads can be fluorescent.

As the concentration of calibration heads in both the test sample and the isotype control are identical, or approximately identical, the calibration heads can be used to ensure that flow cytometry readings are comparable. In particular, the calibration beads can be used to ensure that results are based on approximately the same volume of the test samples. For example, the flow cytometer can be programmed to accumulate readings until a certain number of calibration beads are detected.

As used herein, “approximately the same concentration of beads” is used to refer to approximately the same concentration of beads in the test sample and isotype, control. The difference in the concentration of calibration beads present in the two solutions can be no more than 10%, no more than 5%, no more than 2%, no more than 1%, no more than 0.5%, or no more than 0.25%. One method of achieving this level of accuracy between samples is the use of automated counting techniques or flow cytometry tubes that are pre-loaded with a specified quantity of calibration beads, such as TRUCOUNT flow cytometry tubes marketed by Becton Dickinson.

As used herein, “linked” is used to refer to both direct binding and indirect binding, such as where an immunoglobulin that includes a fluorescent molecule binds to a monoclonal antibody that selectively binds to a protein present on CNPs. As used herein, “binding” refers to direct binding between two materials. Thus, in the above example, the immunoglobulin is linked to the CNP, but not bound to a CNP, but the immunoglobulin binds to a monoclonal antibody that is hound to a CNP.

As used herein, a marker is “specifically linked to CNPs” when the linkage between the marker and the CNPs will not result in a linkage to other particles in the sample. As used herein, a monoclonal antibody “specifically hinds to CNPs” when the monoclonal antibody binds to CNPs, but. not to other particles in the sample. As used herein, “aggregation” and “aggregates” include all forms of aggregation of CNPs to one another including, but not limited to, fusing, agglomeration, etc., whether alone or in combination.

The analysis of the flow cytometer results can include analyzing a plot of side scatter and forward scatter results from the flow cytometer evaluation of the test sample. The plot can be a log-log plot of side scatter and forward scatter results. The plot can be a HA histogram.

The selective linkage can include a monoclonal antibody that specifically binds to the CNPs. The selective linkage can include an antibody that specifically binds to the monoclonal antibody. The selective linkage can include a monoclonal antibody that specifically binds to the CNPs and an antibody that specifically binds to the monoclonal antibody.

The test sample can be produced by creating a first solution comprising CNPs isolated from a biological source, a buffer solution, and a monoclonal antibody that specifically binds to CNPs. The first sample can be incubated for a period of sufficient duration for the monoclonal antibody to bind to the CNPs in the first sample. Next, the test sample can be created by adding a marker to the first sample, where the marker comprises a fluorescent molecule and the marker specifically binds to the monoclonal antibody. The CNPs isolated from the biological source can be obtained from blood, bodily exudates, abscess fluids, cells, tissue, extracted tissue, and combinations thereof.

The monoclonal antibody can be an 8D10 monoclonal antibody (also “8D10”). The marker can include an antibody conjugated to a fluorescent, molecule, where the antibody can be produced against the monoclonal antibody.

The 8D10 monoclonal antibody is a mouse antibody that specifically binds to the surface of calcified nanoparticles. The fluorescent marker can be a fluorescent molecule, such as fluorescein isothiocyanate (FITC), conjugated to an antibody that binds to the monoclonal antibody specific for CNPs. The fluorescent marker can be an immunoglobulin conjugated to a fluorescent molecule. For example, where an 8D10 monoclonal antibody is used to specifically bind to the CNPs, the fluorescent marker can be goat anti-mouse fluorescein isothiocyanate (Gam-FITC). An excess of the fluorescent marker can be added to both the test sample and the isotype control.

An isotype control sample can be produced by substituting the monoclonal antibody that specifically binds to CNPs for another monoclonal antibody that is nonspecific for CNPs or other proteins that may be present in the test sample. An isotype control of an 8D10 test sample may be identical to the test sample except that the 8D10 monoclonal antibody is substituted by a non-specific binding mouse IgG1 antibody. For example, the non-specific binding mouse IgG1 antibody can be reactive against a synthetic hapten but not CNPs or any known human proteins.

Prior to creating the first solution, a liquid containing CNPs from the biological source can be filtered through a 0.2 micron filter. The filtrate from the filtering process can be used to create the test sample.

The isotype control can be produced by creating a control precursor comprising CNPs isolated from a mammalian subject, a buffer solution, and a monoclonal antibody that is non-specific for CNPs. The control precursor can be incubated for a period of approximately the same duration as the incubating step used to produce the test sample. The isotype control can then be created by adding the marker to the control precursor.

It is to be understood that while the invention has been described in conjunction with the preferred specific embodiments thereof, that the foregoing description as well as the examples which follow are intended to illustrate and not limit the scope of the invention. Other aspects, advantages and modifications within the scope of the invention will be apparent to those skilled in the art to which the invention pertains.

The following Examples are provided as one way to illustrate the invention. It should be understood that the Examples described below are provided for illustrative purposes only and do not in any way define the scope of the invention.

### EXAMPLES

### Materials and Methods

Sources of CNPs

Fetal Bovine Serum (FBS)(Hyclone,Logan Utah), human plasma and an aseptic exudate from a patient with lupus panniculitis were used as sources of CNPs. Blood was drawn by phlebotomy, into tubes, which could contain heparin, EDTA, sodium citrate or other reagents known to prevent clotting (BD, San Jose Calif.) and centrifuged at 500 g for 10 minutes. Plasma was separated from the cell fraction and frozen at −21.1° C. The fetal bovine serum (defined PBS, Hyclone, Utah) used was gamma irradiated (25-40 kGy, 2.5 megarads) and filtered though a 40 nm membrane filter. Cultures of plasma or PBS were established in 25 cm² flasks fitted with a screw cap containing a hydrophobic filter (BD, Franklin Lakes, N.J.), which was covered with a layer of parafilm and in either 15 ml and 50 ml polypropylene tubes with the caps tightened (BD, Franklin Lakes, N.J.). The samples were diluted one-quarter (¼) in RPMI 1640 (Sigma, St Louis Mo.) or in serum free media (Ultraculture, Lonza, Walkersville, Md.) and incubated in a humidified incubator at 37° C., 5% CO₂. Alternatively, samples were filtered through a 0.2 micrometer filter (Millex, Millipore, Bedford Mass.). They were then diluted one-quarter (¼) in RPMI 1640 and incubated, as above. The biofilm produced by CNPs grown in flasks was harvested by using a rubber spatula and then homogenizing the sample by repeated pipetting. An advantage of using polypropylene tubes was that the CNPs did not attach and could be readily harvested for analysis. However, they replicated and formed crystals and aggregates in a fashion similar to CNPs grown in flasks. When cultured in flasks, particles could be easily observed using phase microscopy (Olympus CK2, Japan) and focusing on the monolayer using 400×.

Flow Cytometry

Samples were analyzed on a FACSCalibur flow cytometer (BD Biosciences, San Jose Calif.) equipped with a 15 mW air-cooled 488 nm argon laser for excitation of FITC. Cytosettings were established for the forward scatter (FS) Log at 00 and side scatter (SS) Log at 350.

Threshold levels were set at 50 for both FS and SS parameters to reduce the number of events due to background. The first fluorescent channel (FL1) was set in log mode. The machine was set on a low rate of flow and acquisition was always stopped at 30 seconds or, when indicated, at a set number of events on the region of an internal standard (6 μn beads). When indicated, 6 μm fluorescent beads (Polysciences, Warrington Pa.) that had been thoroughly vortexed and sonicated in a water bath for 10 minutes, were used as an internal standard. The beads were diluted 1/10 in phosphate buffered saline (or “PBS”) with Ca and Mg to a final dilution of 2×10⁵ beads/ml and this suspension was used to re-suspend each sample before transferring to the flow tubes. The samples were thoroughly vortexed before acquisition.

Analysis Using Microbeads (25 nm, and 6 um in diameter)

Fluorescent microbeads, 25 nm in diameter (cat. #F8760 Invitrogen, Carlsbad Calif.), were diluted 1110× and 1/100× and 1/1000× in sterile PBS. PBS without beads was also analyzed for background event count. The bead solutions were analyzed using flow cytometry in a double parameter dot plot (FSLog and SSLog). Acquisition was stopped after 30 seconds or another pre-determined time interval, volume of sample, or number of calibration beads.

FBS (1 ml) was diluted one-quarter (¼) in RPMI 1640 and incubated, for one month in flasks. Flasks were scraped and the CNPs containing media was collected. Three dilutions, 1/10, 1/100 and 1/1000 of this solution of CNPs in PBS were prepared. Microbeads (6 μm, Polysciences, Warrington, Pa.) (100 μl sample) were added to 900 μl of each of the CNPs dilutions so that the heads were at a final concentration of 2×10⁵ heads/ml in PBS with Ca and Mg.

HCl treatment

A heavily calcified culture of CNPs from the panniculitis exudate was separated into four aliquots of 0.5 ml each and centrifuged at 19,000 g for 30 minutes. The supernatant fluid was discarded. The pellets were resuspended in diluted in sterile PBS, to the following concentrations: 1N, 0.5N, 0.2 N, 0.1N, 0.05N. The suspensions were vortexed and incubated overnight at room temperature. They were then centrifuged at 19,000 g for 30 min and resuspended in 0.5 ml of sterile PBS and transferred to flow cytometry tubes.

Analysis of CNPs using 8D10 monoclonal antibody

Samples of cultured CNPs (250 μl) were collected and diluted one-quarter (¼) in sterile PBS with Ca and Mg (Sigma, St Louis Mo.). The diluted samples were centrifuged at 19,000 g for 45 minutes and the supernatant fluid was discarded. The pellets were re- suspended in a solution of 8D10 monoclonal antibody (10 μg/ml final concentration) in PBS with Ca and Mg. The 8D10 monoclonal antibody is an Immunoglobulin 1 (IgG1) isotype. Therefore, as a control for non-specific binding, a mouse IgG1 antibody, reactive against a synthetic hapten was employed, at 20 μg/ml. The negative control as provided does not react with any known human proteins (LabVision, ThermoFisher Scientific, Fremont, Calif.). Three other mouse IgG1 monoclonal antibodies, specific for lymphocyte surface proteins, diluted at 20 μg/ml in PBS w/Ca/Mg, were analyzed for their reactivity as well. Samples were vortexed and incubated overnight at 4° C. A secondary antibody, goat anti-mouse IgG1 conjugated to fluorescein (Gam-FITC) (Pierce, Rockford Ill.) was added at 75 μg/ml final concentration in PBS with Ca and Mg. Samples were incubated in the dark in secondary antibody for one hour at approximately room temperature (RT). They were then diluted in 500 μl of PBS with Ca and Mg and transferred to flow cytometry tubes. A sample stained with secondary antibody alone was used as a control for non-specific binding of the conjugated antibody. Five hundred microliters of a reagents alone (8D10 at 10 μg/ml and Gam-FITC at 75 μg/ml final concentrations) solution in PBS, were also used to evaluate the number of events due to background.

Ultracentrifugation of FBS Samples

Ten ml samples of defined FBS (40 nm filtered, gamma irradiated) were 0.2 μm filtered and the filtrate was centrifuged at 40,000 g in an ultracentrifuge (Beckman L8M, Palo Alto, Calif.) for 1 hour, at 4° C. The supernatant fluid was collected, diluted one-quarter (¼) in RPMI 1640, and an aliquot was incubated at 37′C for 5 days. Aliquots of the same PBS sample, not ultra-centrifuged, were also treated in the same way. As a control for time 0, aliquots were kept at −20° C. and were thawed prior to analysis, simultaneously with the incubated samples, in the flow cytometer.

Analysis of a Lupus Panniculitis Exudate Sample Over Time

A CNP culture from a systemic lupus erythematosus (SLE) patient's plasma that had many, highly motile forms, was diluted 1:4 in RPMI with 20% FBS (gamma irradiated, 40 nm. filtered) and was filtered through a 0.2 μm filter. The filtrate was incubated in a polypropylene tube for 6 days. Three 250 μl aliquots were obtained from the culture every 12 hours and frozen at −20° C. As a negative control, the same media with 20% defined FBS, but with no CNP innoculum, was also incubated and aliquots extracted in triplicate, and frozen, in identical manner. The aliquots were all thawed at the time of the flow cytometry assay and read simultaneously. A suspension of 6 nm microbeads was vortexed and sonicated, diluted to 2×10⁵ beads/ml in sterile, tissue culture H₂O (Sigma, St Louis, Mo.) and vortexed and sonicated again to obtain a homogeneously dispersed suspension. The samples were diluted one-half (½) in the microbead suspension to a final volume of 500 μl and transferred to flow cytometry tubes.

Cultures of Plasma from SLE Patients and a Healthy Donor.

Samples (2 ml) of plasma from SLE patients and a healthy donor were diluted one-quarter (¼) in RPMI1640 and incubated, or not, at 37° C. for 18 days. Aliquots (1 ml) were frozen at −20° C. Samples were thawed and stained with 8D10 of IgG negative control and Gam-FITC and resuspended in a solution of PBS with 6 μm heads to be used as an internal standard, as indicated above. The samples were transferred to flow cytometry tubes and analyzed in the flow cytometer.

### Results

Placement of CNPs Within a Flow Cytometry FS vs SS Plot Using Beads of Standard Sizes: Identification of Size Range of CNPs Using Microbeads

The use of microbeads of two sizes, 25 nm and 6 μm in diameter, allowed evaluation of the settings on the FSLog vs SSLog, which would encompass the range of sizes of the CNPs and their crystalline aggregates.

Microbeads 25 nm in diameter were utilized at three different concentrations and cytosettings were adjusted so this size of particles was detected in the lower left hand quadrant of a FSLog vs SSLog dot plot. As demonstrated by FIG. 1, the FS vs SS dot plots showed that 22 nm particles could be detected in the flow cytometer, and that the difference in particle concentrations, could be evaluated.

FIG. 1 demonstrates that microbeads approximating the size of many CNPs (25 nm in diameter) can be quantitated and used as an internal standard. Microbead solutions were diluted 1/10, 1/100 and 1/1000, in sterile PBS and analyzed by flow cytometry. The events registered in the determined region (R1) of the FS vs SS plot were lower with increasing dilutions.

The 6 μm beads were then analyzed. The 6 μm beads, because of their larger size, could be excluded from the region where CNPs had been previously detected. A region (R1) was drawn in the FSLog vs SSLog plot, around the 6 μm beads. Acquisition was stopped at 300 events in the region containing the beads, in every case.

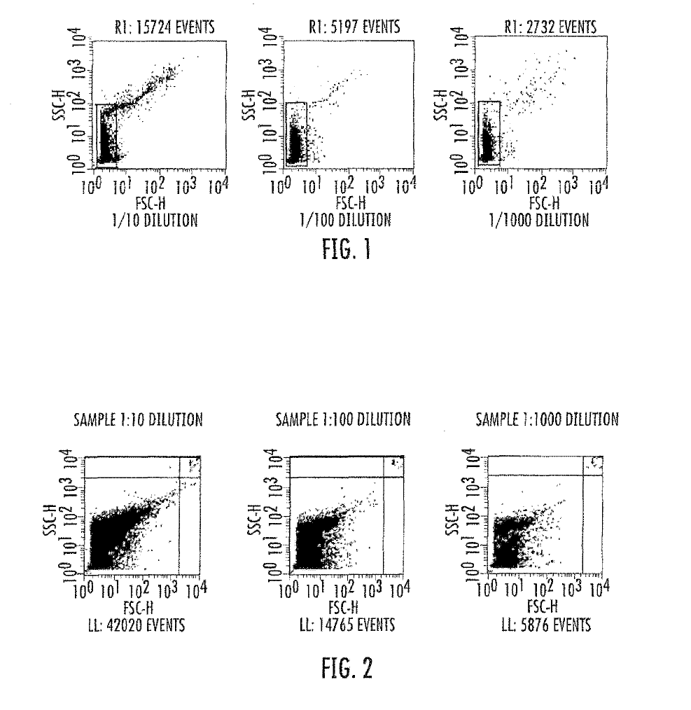

Using the same cytosettings as with the 25 nm beads, the 6 μm beads were detected on the upper right hand quadrant of the FS vs SS dot plot, as shown in FIG. 2. The CNP signal did not overlap with the 6 μm heads signal. As shown in FIG. 2, the number of events in the lower left quadrant of the plot were inversely related to the dilution of CNPs, indicating that the different concentrations of CNPs were comparable from one graph to another, in a significant manner, when they were normalized to a constant number of beads.

In FIG. 2, a CNP sample was diluted to 1/10, 1/100 and 1/1000 in PBS containing 2×10⁵ beads/ml. Acquisition was stopped at 300 events in the 6 μm beads region. The number of events in the lower left quadrant is inversely correlated to the dilution of CNPs.

Different Sizes of CNPs Can Be Discriminated in a FSLog Vs SSLog Histogram.

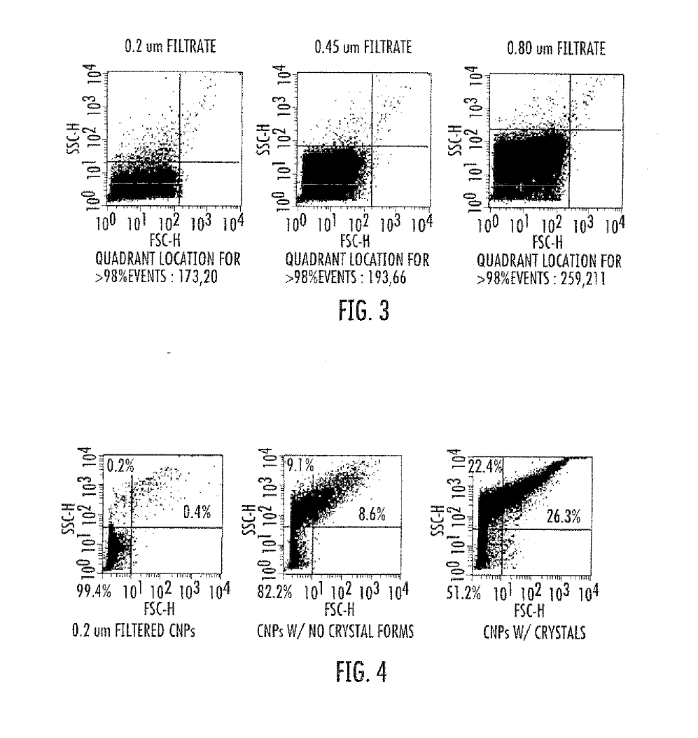

To further characterize CNPS by size, a culture was filtered through ultrafilters of different size pores and analyzed on the flow cytometer. Aliquots (1 ml) of an active culture of CNPs from flasks were filtered through ultrafilters with different sized pores: 0.2 um, 0.45 um, and 0.80 um (Millipore,Billerica, Mass.). The filtrate was transferred to flow cytometry tubes and analyzed in the flow cytometer using the same cytosettings as with the 6 μm microbeads. Acquisition was stopped at 30 seconds. The quadrant lines were used to determine four regions on the FSLog vs SSLog histograms, so that >98% of the events were in the lower left quadrant, and the Quadrant Location numbers were used to show where the different filtrates appeared on the FSLog vs SSLog plots. This indicated where CNPs between 200 nm and 800 nm in diameter, were detected, As demonstrated by FIG. 3, as CNPs aggregate into particles of different sizes, and expand in diameter with time of incubation, these cut-off values are useful to evaluate the approximate size of these particles.

In preparing the FIG. 3 analysis, CNP cultures were tittered through (a) 0.2 μm filters, (b) 0.45 μm filters, and (c) 0.8 μm filters, and the filtrates were analyzed on FSLog vs SSLog plots. The quadrant locations placing >98% of the CNPs events on the lower left quadrant show the different sizes of particles that can be discriminated by flow cytometry.

Samples of a two week old culture of CNPs from plasma were then compared with a culture that contained many large crystalline forms, As can be seen from FIG. 4, the FSLog vs SSLog plots could detect larger crystalline forms in the upper right hand quadrant, indicating they were large in size and very refractive to light. Different types of CNP cultures could be effectively characterized using a double parameter FSLog vs SSLog plot.

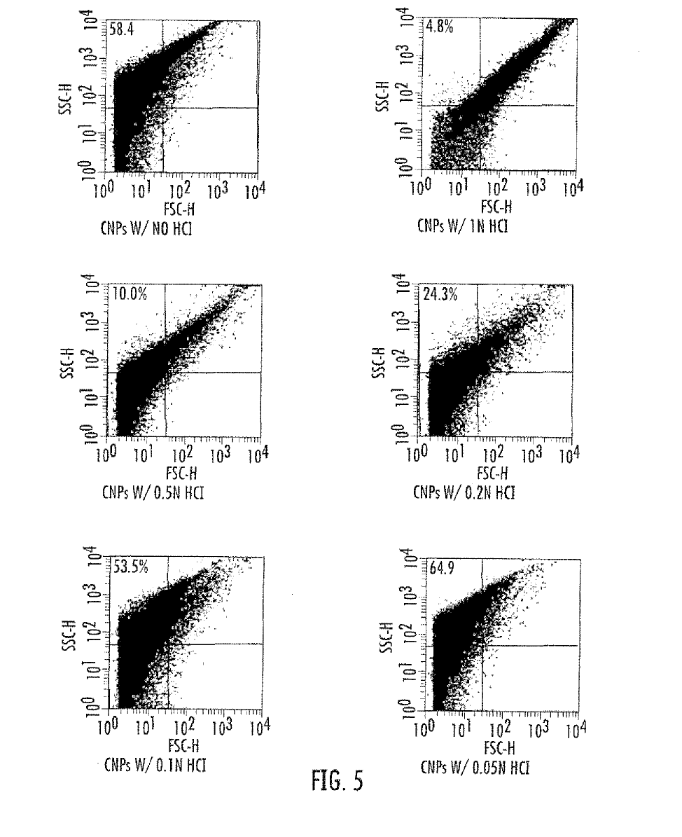

To confirm these findings, aliquots of a heavily crystallized culture were treated with different concentrations of HCl and analyzed in the flow cytometer. It could be seen that the FL1 histograms of the HCl treated samples lost the signals due to small, highly scattering particles, corresponding to the demineralization of these small particles. An overnight treatment of 1N HCl did not dissolve all the larger crystals. As shown in FIG. 5, when the concentration of HCl was lower, there were more particles in the upper left quadrant, and the dot plots resembled those of the untreated samples.

FIG. 4 depicts a comparison of the FSLog vs SSLog dot plots of CNPs from a 0.2 μm filtered sample, a two week old culture sample, containing small motile forms, and a sample from an older, heavily crystallized culture.

FIG. 5 shows a comparison of the FSLog vs SSLog dot plots of CNPs from a heavily calcified culture that were untreated or treated with HCl at different concentrations.

The 8D10 Monoclonal Antibody Can Be Used to Differentiate Positive And Negative Samples of CNPs by Flow Cytometry.

CNPs in Non Gamma-Irradiated Fetal Bovine Serum

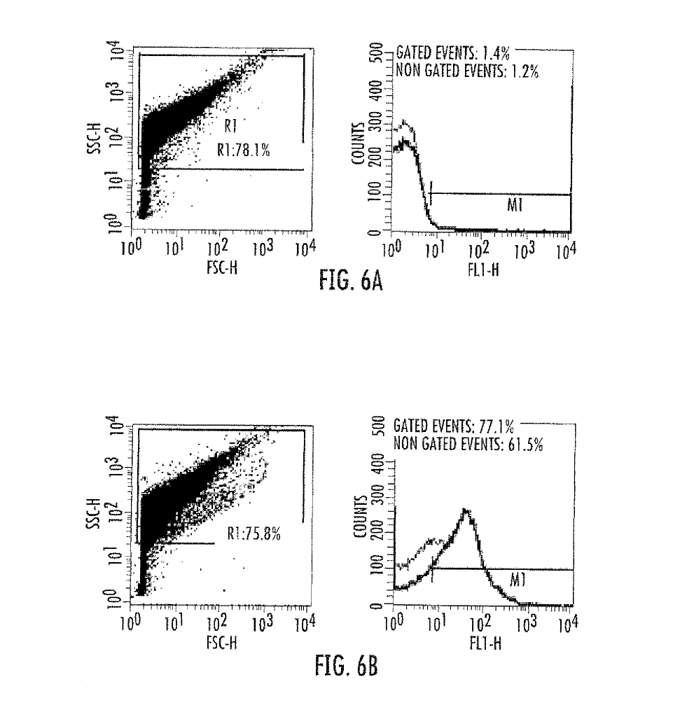

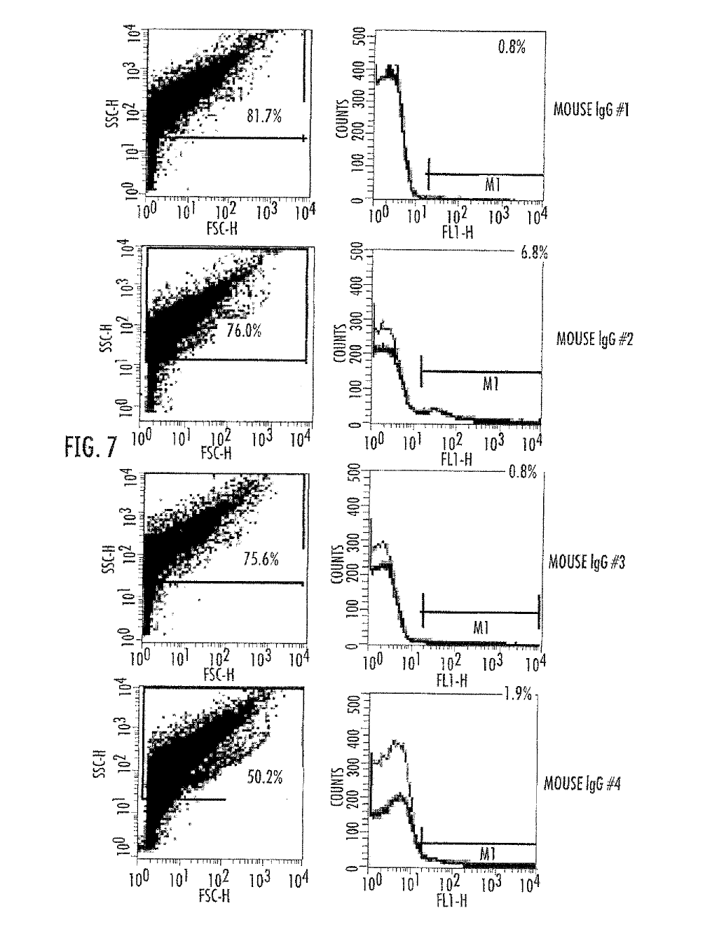

A sample of PBS that had been incubated in RPMI at a one-quarter dilution for 1 month at 37° C. was analyzed by flow cytometry. CNPs present in the PBS sample, when stained with a monoclonal antibody of the same isotype as 8D10, that was specific for a synthetic hapten, and the secondary antibody, goat anti-mouse IgG1-FITC(Gam-FITC), did not show any events in the positive region of FL1, as shown in FIG. 6A. As shown in FIG. 6B, a similar sample, stained with the 8D10 monoclonal antibody (IgG1 istoype) and Gam-FITC, showed a high percentage of events in the positive region of PILL

A region (R1) was established on the FSLog vs SSLog dot plot to evaluate the differences in staining according to the size and light scattering characteristics of the CNPs; the FL1 histograms were gated, or not, in the R1 region. A region (Ml) was determined on the FL1 histogram to exclude those event counts due to non-specific binding of the non- specific mouse IgG1 antibody.

The FL1 histograms shown in FIG. 6 are an overlay of the gated (dark line) and the ungated events (light line) in the R1 region. It is apparent that the particles with the lowest side scatter (also “SS”) are the negative ones for 8D10, corresponding to very small, particles that do not scatter light. These assays were repeated at least three times with different PBS samples, with similar results each time.

In FIGS. 6A & B, FBS samples were analyzed for their reactivity with 8D10 monoclonal antibody. FIG. 6A depicts a PBS sample stained with a negative control IgG1 antibody and goat anti-mouse antibody conjugated to fluorescein isothiocyanate (Gam-FITC). FIG. 6B shows a PBS sample stained with 8D10 and Gam-FITC. The RA histograms are overlays of the ungated events (thin line), and events gated on R1 (thick line).

In order to ensure that the negative control antibody was effective, three other mouse IgG1 antibodies of irrelevant specificities were assayed. As shown in FIG. 7, none of them showed positive staining on FL-1.

In FIG. 7, CNPs cultures were stained with negative control mouse IgG1 and Gam-FITC(#1) and three different mouse IgG1 monoclonal antibodies of irrelevant specificity (#2,#3,#4). On the FL1 histograms, the thin line corresponds to non-gated events, the thick line to events gated on R1.

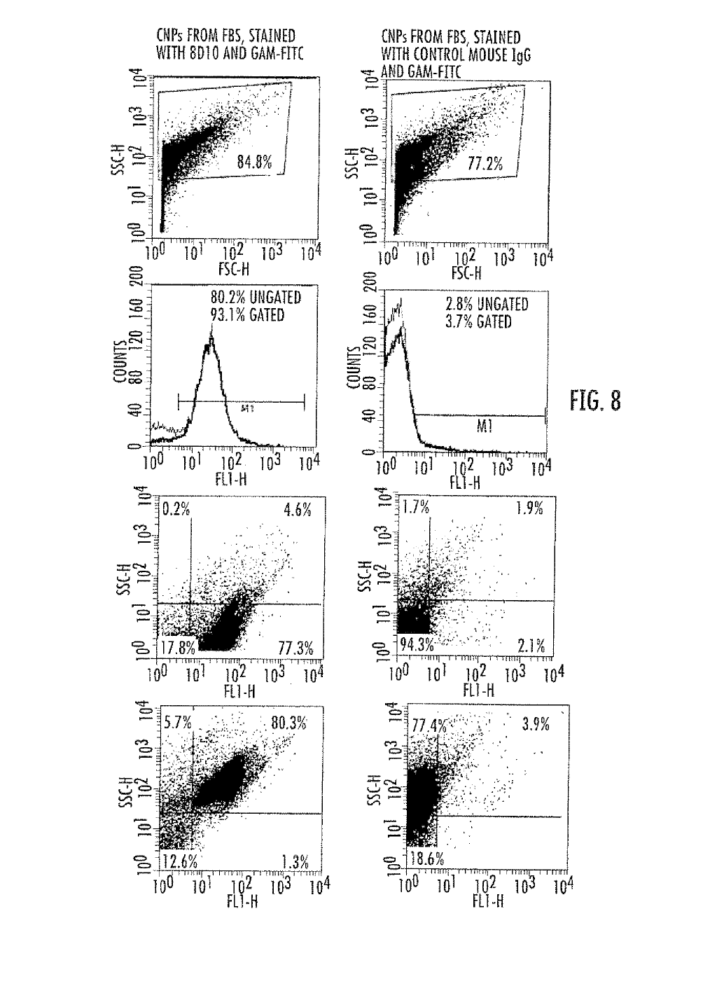

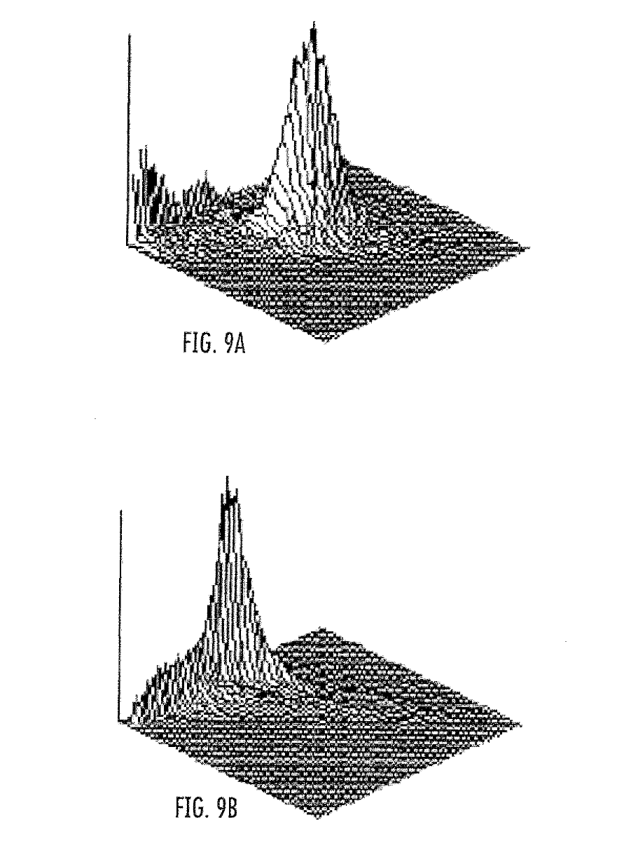

In FIGS. 8 and 9, CNPs obtained from a PBS sample that was not cultured in RPMI, also stained positive for 8D10 monoclonal antibody and not for the isotype control. In FIG. 8, the SS versus FL1 and FS versus FL1 dot plots are not gated on R1. Particles that stained positive for 8D10 (on the FL1 positive region), are also the ones with low FS values (very small) yet high in light side scattering which would correspond to small, highly refractive particles.

In FIG. 8, the FL1 histograms were gated (dark line), or not (light line), on R1 of the ES versus SS plot. The SSLog versus FL1 and FSLog versus FL1 dot plots show that particles staining positive for FL1 are also high in SSLog (90° light scattering) and low in FSLog (size).

FIGS. 9A and 9B show three dimensional plots of SSLog versus FL1. FIG. 9A depicts a PBS sample stained with 8D10, while FIG. 9B depicts a PBS sample stained with mIgG. This plot shows particles that have high FL1 fluorescence, also exhibit high levels of side scatter light dispersion.

Effect of ultracentrifugation of FBS samples, on the flow cytometric analysis of CNPs using 8D10 monoclonal antibody.

FBS samples that had been ultracentrifuged, and a similar sample that had not been ultracentrifuged, were diluted in RPMI and incubated for 5 days at 37° C. They were subsequently analyzed for 8D10 reactivity as described above in Materials and Methods.

Comparing the histograms at time 0 with those after 5 days of incubation at 37° C. the percentages in the positive region of FL1 increase significantly (32.5% to 86.6% in the non-centrifuged sample and 20.7% to 58.2% in the ultra-centrifuged sample).

After 5 days of incubation, the non-centrifuged samples showed a greater percent of 8D10 positive events (86.6%) than the ultracentrifuged samples (58.2%). In the FL1 versus SS plot, the percentage of particles positive for FL1 with high SS is 79% in the centrifuged sample, and only 26% in the non-centrifuged sample. In the FL1 versus FS dot plot, the small, yet positive samples on the non-centrifuged sample were 71.4% versus 22% in the centrifuged sample.

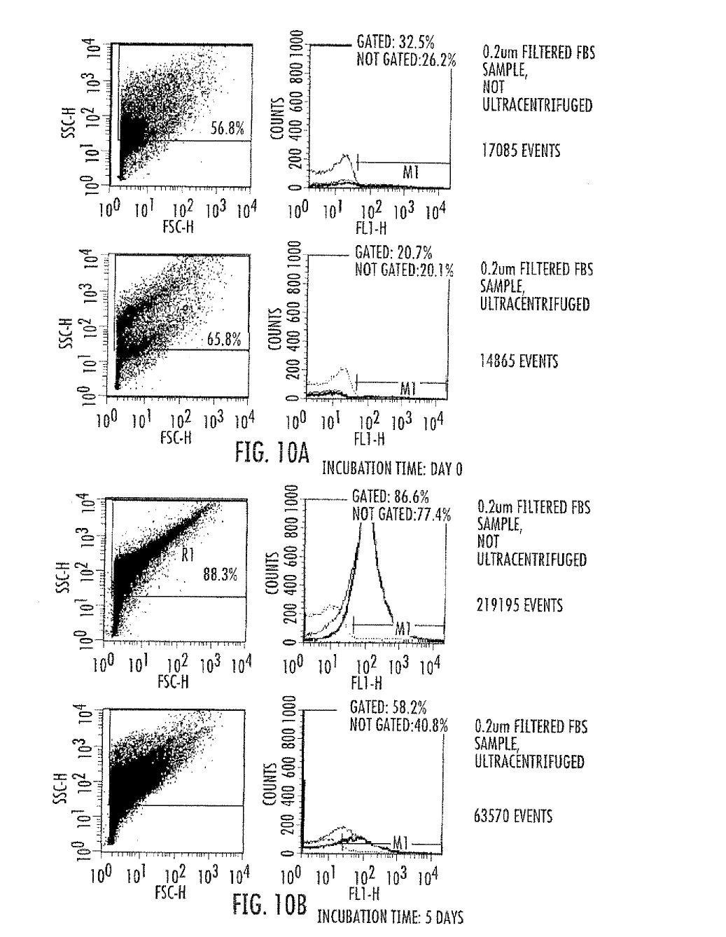

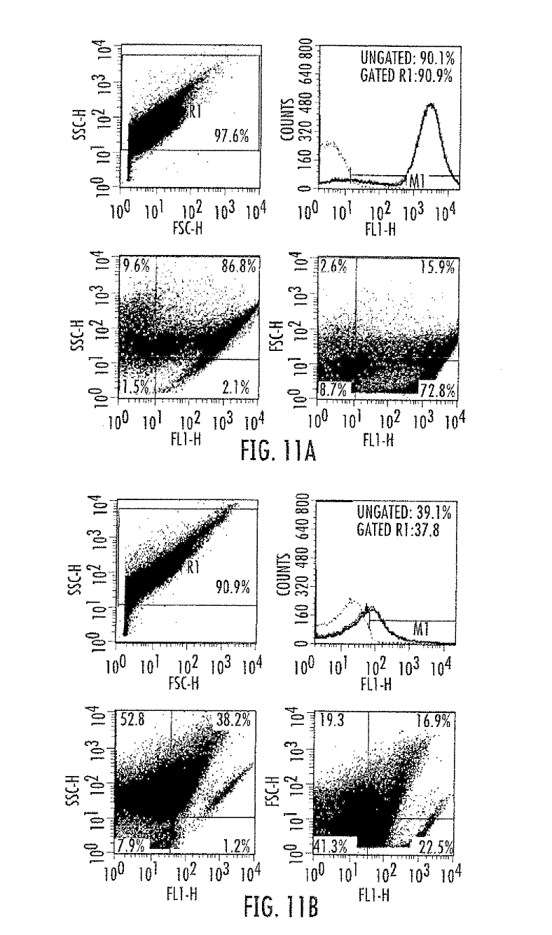

Based on FIGS. 10A and 10B, it could be concluded that ultracentrifugation at 40,000 g for 1 hour greatly diminished the amount of 8D10 staining particles but did not eliminate them, as after 5 days of incubation, the percent of 8D10 stained CNPs increased regardless of whether they were centrifuged or not.

FIG. 10A depicts results for samples before incubation, while FIG. 10B depicts results for samples after 5 days of incubation. In FIGS. 10A and 10B, the dashed line is the isotype control, the thin line corresponds to non-gated events, the thick line corresponds to events gated on R1.

CNPs in Human Plasma

Healthy donor's and SLE patients' plasmas were analyzed by flow cytometry, for the presence of 8D10 positive particles. Plasmas that were kept at −20° C. were thawed, and immediately analyzed in the flow cytometer or they were diluted in RPMI 1640 (¼ dilution) and incubated at 37° C. for two weeks, in flasks, before analysis.

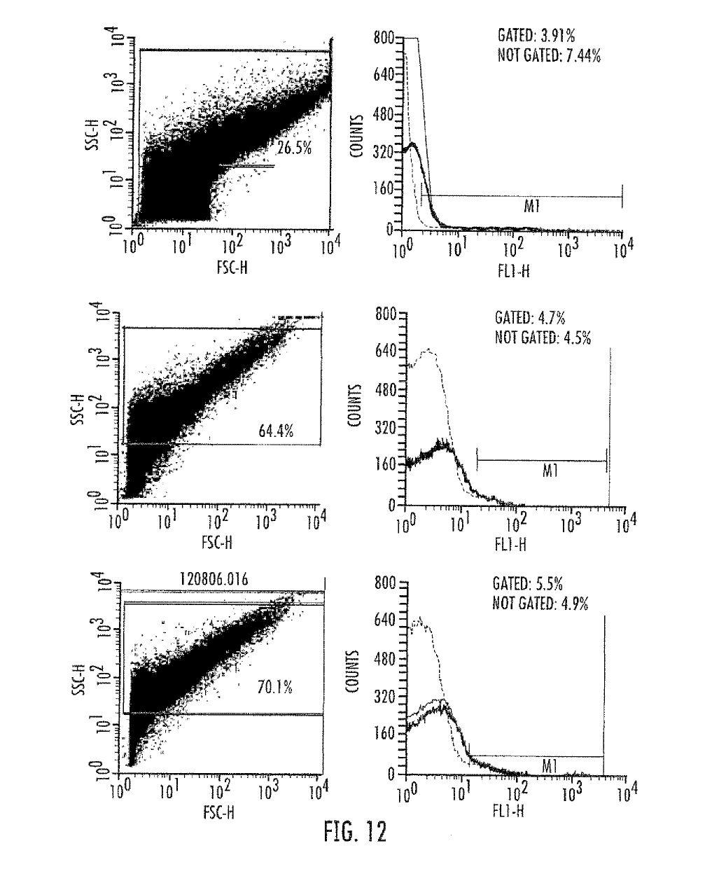

FIGS. 11A and 11B show results for samples stained with 8D10 or with mouse IgG I and counterstained with secondary antibody labeled with FITC (Gam-FITC), and were analyzed in the flow cytometer as described above.

In FIGS. 11A and 11B, a region (R1) was established in the FS vs SS dot plot to exclude particles with low SS, and histograms were gated, or not, on this region. The HA histograms are an overlay of the gated (dark line) and the ungated events (light line) on R1.

A positive and a negative plasma sample were analyzed on three different occasions, and they were consistently positive or negative, respectively. Eleven out of twelve samples of healthy donors were positive for 8D10 monoclonal antibody staining. All the lupus patients' samples (10/10) were positive for 8D10 antibody staining. As shown in FIG. 12, the negative sample was consistently 5% or less, on FL-1 with respect to the non-specific control.

FIG. 11A shows results from recently thawed plasma, while FIG. 11B shows results for plasma incubated for 14 days at 37° C. In the FL-1 histogram in FIGS. 11A & B, the dashed line corresponds to the isotype control, the thin line corresponds to non-gated events, the thick line corresponds to events gated on R1 of the FS versus SS plot.

FIG. 12 depicts the analysis of three different samples of a healthy donor's uncultured plasma, using 8D10 monoclonal antibody. On the FL-1 histogram of FIG. 12, the dashed line corresponds to the isotype control, the thin line corresponds to non-gated events, and the thick line corresponds to events gated on R1.

Analysis of CNPs in a Lupus Panniculitis Exudate

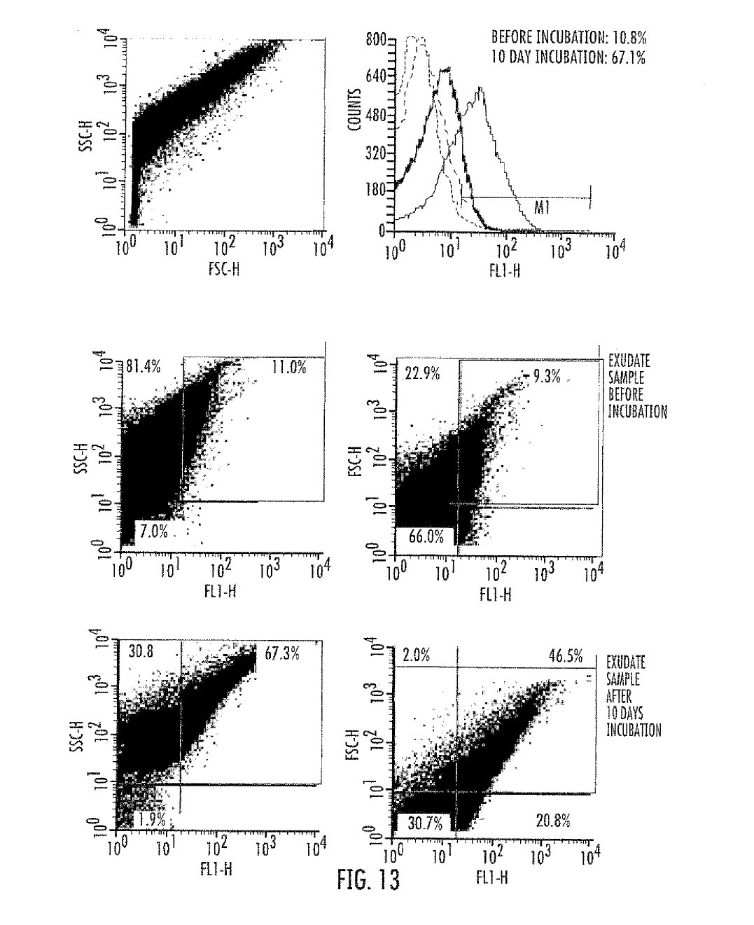

A sample from a lupus panniculitis exudate was stained with 8D10 monoclonal antibody or isotype control and analyzed by flow cytometry. Aliquots were taken before and 14 days after incubation in serum free media, at 37° C. The sample before incubation showed a very low percentage of 8D10 positive events, but this percentage dramatically increased after the incubation period. As shown in FIG. 13, when analyzing the FS versus HA and SS versus FL1 plots, the increase in HA positive particles due to highly scattering particles is evident. On the FL1 histogram of FIG. 13, the dashed line corresponds to the isotype controls, the thin line corresponds to events from the incubated sample, the thick line corresponds to events from the sample before incubation,

Flow Cytometric Analysis of CNP Cultures, at Different Times of Incubation:

A CNP culture, from an SLE plasma sample that had been analyzed by flow cytometry, found to be positive for 8D10 and observed by light microscopy to have many, highly motile forms, was used in this assay. Aliquots were obtained and processed as indicated in Materials and Methods, using a solution of 6 μm beads in PBS as an internal standard.

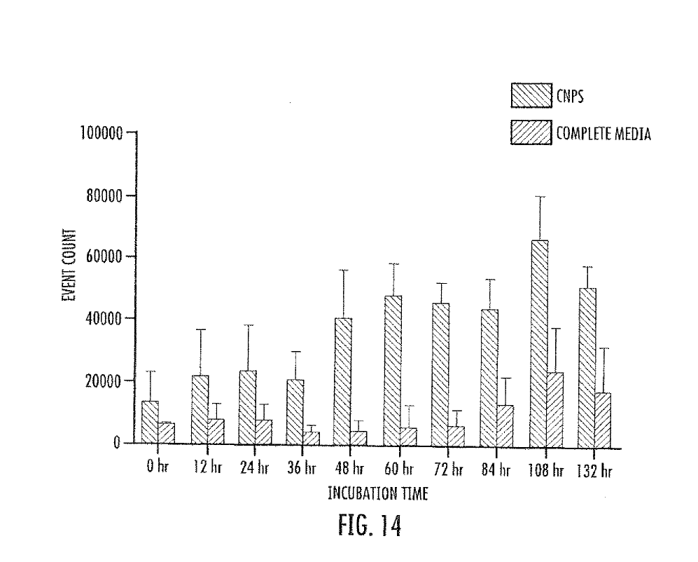

Samples were examined in the flow cytometer at a low rate of flow, and acquisition was stopped at 1000 events on the 6 μm bead region drawn on the FS vs SS scattergram. Only total events on the lower left quadrant, were recorded. Three different aliquots were analyzed for each time point, and the bars indicate the average and standard deviation recorded for each time point. As a control, an aliquot of complete media, with no CNPs was likewise incubated and analyzed, in triplicate, for each time point. As seen in FIG. 14, the number of events recorded in the inoculated sample becomes larger over time. The number of events of the non-inoculated media sample remains constant except for the numbers recorded after the third day, where there is a slight increase, possibly due to the effect of CNPs in the FBS.

FIG. 14, depicts the Total number of events on FS vs SS dot plots, from aliquots of a CNPs culture and the corresponding media control samples, taken at different time points of incubation. Cultures were incubated at 37° C. for 6 days. Aliquots were taken every 12 hours for the first three days, and every 24 hr for the following three days. Acquisition was always stopped at 1000 events in R1 (6 μm beads region).

Flow Cytometric Analysis of SLE Patients' Plasma at Different Times of Incubation, Using 8D10 Monoclonal Antibody.

Freshly thawed plasma samples from two different SLE patients (#1 and #2), were diluted one-quarter (¼) in complete media and incubated in polypropylene tubes for 18 days. Aliquots were taken every 3 days and frozen at −20° C. Another tube containing complete media alone was likewise incubated and aliquots extracted at the same time points as the samples. The aliquots were thawed and analyzed simultaneously, using 8D10 monoclonal antibody, and 6 μm beads were used as an internal standard for semi-quantitative purposes.

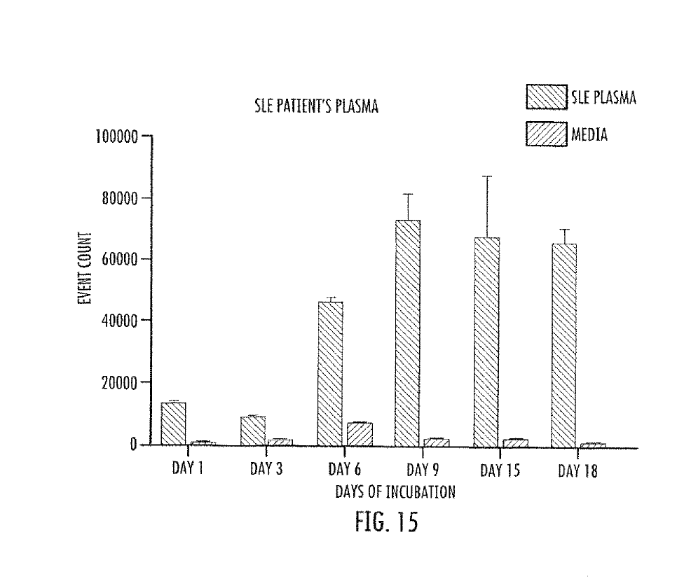

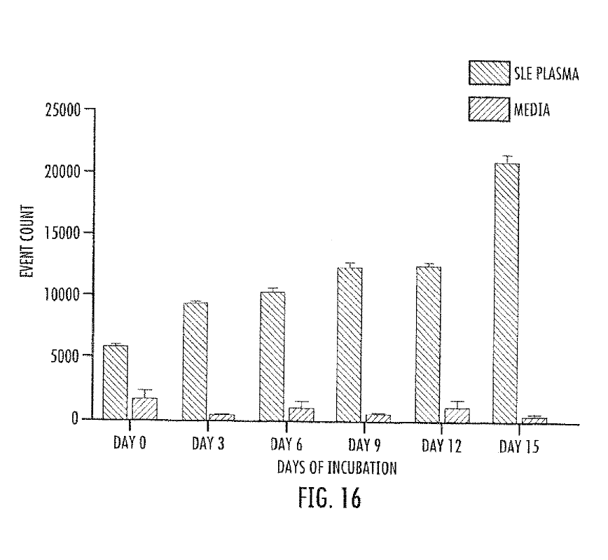

FIG. 15 and FIG. 16 show that there is an increase in 8D10 positive particles over time of incubation and the media alone controls do not increase over time, in both patients' samples. This assay was repeated two other times with SLE patients and with a healthy donor, with similar results (data not shown).

As can be seen in FIG. 15, the number of events on FL-1 channel increases with time of incubation. The number of events on FL1 channel (8D10 monoclonal antibody positive region) registered in aliquots taken at different times of incubation of an SLE donor's plasma(#1) diluted one-quarter (¼) in RPMI 1640. in FIG. 15, the controls are the complete media incubated without the patient's plasma.

As can be seen in FIG. 16, the number of events on FL-1 channel increases with time of incubation. Number of events on FL1 channel (8D10 monoclonal antibody positive region) registered in aliquots taken at different times of incubation of an SLE patient's plasma (#2), diluted one-quarter (¼) in RPMI 1640.In FIG. 16, the controls are complete media incubated without the patient's plasma.

Effect of Incubation of Healthy Donors' And SLE Patients' Plasmas When Analyzed Simultaneously in the FL1 Histogram.

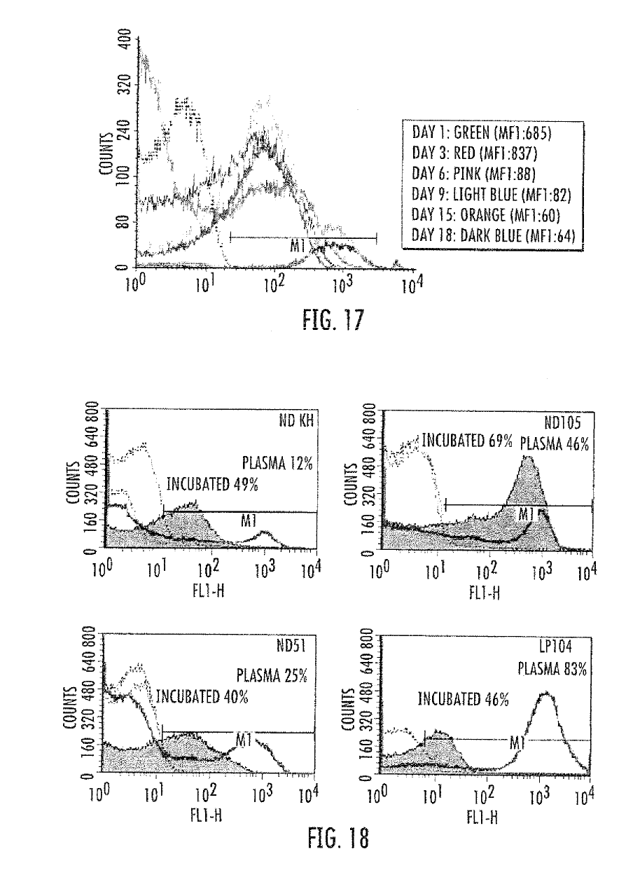

Plasma samples were compared before and after incubation at 37° C. for several days, for their reactivity to 8D10 monoclonal antibody. Comparing the overall 8D10 reactivity, samples did not become negative with culture. What was noticeable was that in three out of seven healthy donor samples, and three out of four SLE plasma samples analyzed, there was a shift to the left in the FL1 histogram curves. As shown in FIGS. 17 and 18, this is verified by comparing the peak channels and the mean fluorescence intensity in the respective FL1 histograms. This result was also obtained on a plasma sample that was filtered through a 0.2 μm filter just before incubation. In this case the overall number of events is appreciably lower, due to the 0.2 μm filtration which excluded the larger aggregated CNPs.

The data in FIG. 17 can be used to analyze the shift towards lower mean fluorescence intensities (MFI) in. the positive region for 8D10 monoclonal antibody. In FIG. 17, an SLE patient's plasma was analyzed for 8D10 reactivity, using samples taken at the indicated times of incubation. The dotted line in FIG. 17 corresponds to the isotype control.

FIG. 18 shows the shift toward lower mean fluorescence intensity on FL-1. Three healthy donors' and one SLE patient's plasmas show a shift towards lower mean fluorescence intensity after seven days of incubation at 37° C. In FIG. 18, the red lines correspond to freshly thawed plasma and the green area corresponds to plasma analyzed after incubation.

Characteristics of Size and Light Scattering Properties of CNPs Stained with 8D10 Monoclonal Antibody.

The flow cytometry data corresponding to plasma that had been incubated for 18 days was analyzed on FS vs FL-1 and SS vs FL-1 double parameter plots, and the results were compared with those of the same, but freshly thawed plasma.

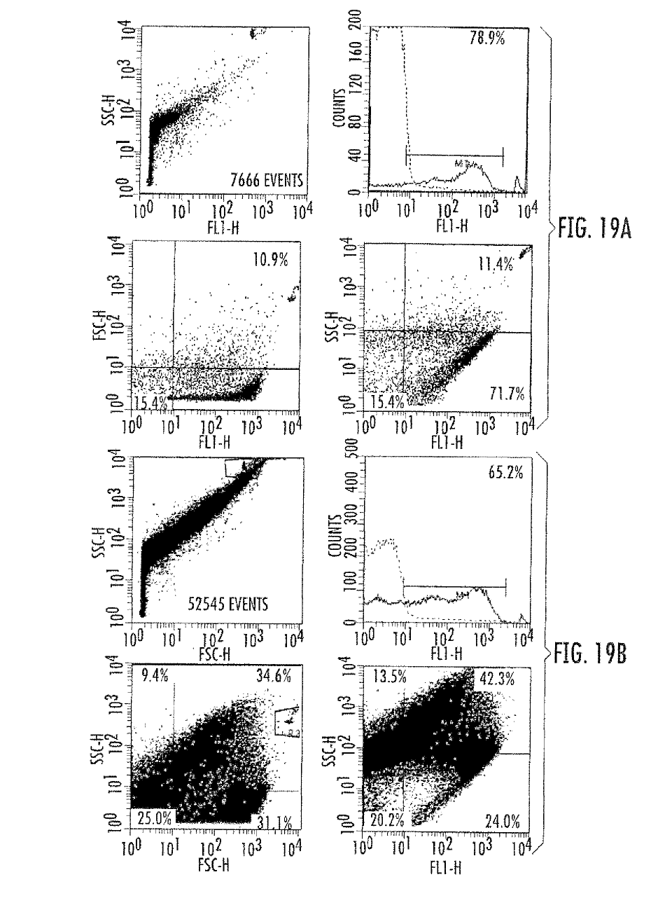

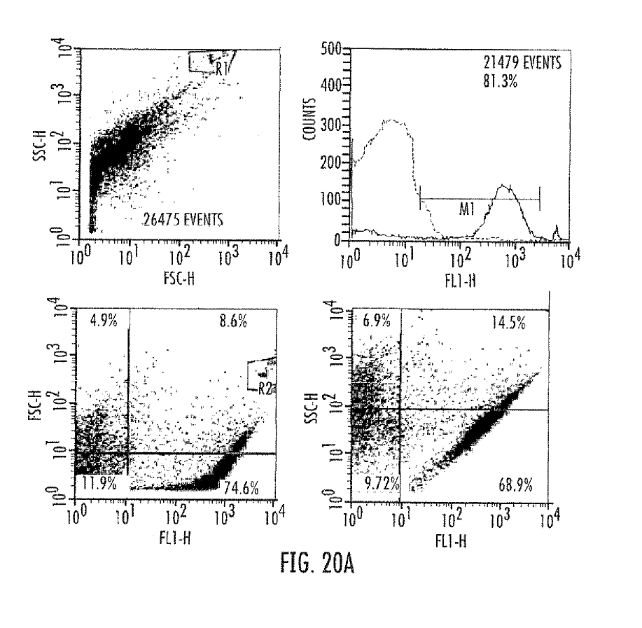

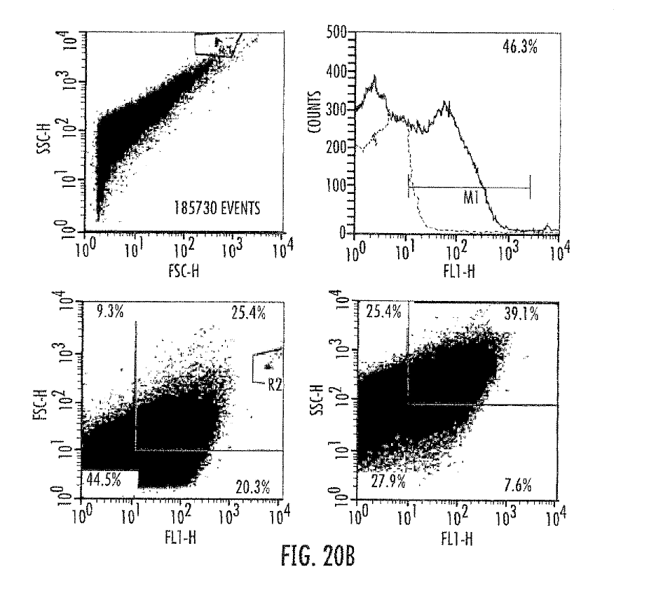

In FIG. 19 and FIG. 20, the CNPs from two SLE patients plasmas show that after incubation, the number of particles increases, and they become larger (higher FS percentages) and increase in light scattering properties (higher SS percentages) while at the same time show a lower mean fluorescence intensity, when compared with freshly thawed plasma.

FIGS. 19A and 19B show the characteristics of size and light scattering properties of CNPs stained with 8D10 monoclonal antibody. FIG. 19A shows flow cytometric analysis of an SLE patient's plasma (#1), before incubation, whereas FIG. 19B shows a flow cytometric analysis of an SLE patient's (#1) plasma after 18 days of incubation at 37° C.

FIGS. 20A and 20B show characteristics of size and light scattering properties of CNPs stained with 8D10 monoclonal antibody. FIG. 20A shows flow cytometric analysis of a SLE patient's plasma (#2) before incubation at 37° C., whereas FIG. 20B shows flow cytometric analysis of an SLE patient's plasma(#2) after 18 days of incubation at 37° C.

### DISCUSSION

CNPs were obtained from different sources and analyzed by flow cytometry. As these particles were extremely small, microbeads of known sizes were used to ensure that the data recorded by the flow cytometer was accurate.

Particles as small as 25 nm could be detected with the flow cytometer, and the differences in 25 nm head concentration discriminated above the background noise when using sterile reagents. Six micrometer beads could be used as an internal standard to compare numbers of particles between two samples analyzed under the same conditions, as they were large enough that a region drawn around them, would exclude them from the events due to the CNPs sample the effectiveness of this internal standard for a semi-quantitative volumetric measure of CNP load was verified.

In the case of the flow cytometry technique, 8D10 monoclonal antibody was efficient as it specifically could detect a protein present in CNPs, when compared with an equivalent non-specific mouse IgG1, used as an isotype control. Furthermore, through flow cytometry it was possible to differentiate the 8D10 positive events in large, highly calcified particles from smaller, less calcified ones, as evidenced in the FLI vs SS and FL1 vs FS dot plots. Thus, flow cytometry proved to be a very useful tool for characterizing the different cultures.

Even though the PBS had been filtered through 40 nm and gamma irradiated at 25 to 40 Kgrey, particles that were positive for 8D10 were still present when analyzed by flow cytometry. Ultracentrifugation at 40,000 g for 1 hour diminished the percentage of 8D10 positive particles, when compared to the same, non-centrifuged sample; but did not eliminate them. The percentage of positive particles increased after 5 days of incubation. Thus, even after gamma irradiation, 40 nm filtration and posterior ultracentrifugation, there were still 8D10 positive CNPs that were capable of increasing in number, when cultured in RPMI for 5 days at 37° C.

It has been reported by Kajander that about 15% of healthy donor plasmas are positive for 8D10 when used in an ELISA. Using this method, it was surprising that the percentage of healthy donor plasmas, positive for 8D10, was 92% (12/13 positive plasmas). Plasmas that were negative by ELBA were positive for 8D10 and this coincided with the fact that these same plasmas, when cultured for one month in RPMI with no PBS, were visually positive by light microscopy (400×).

When analyzing the same plasma sample before and after incubation, the positive signal still remained, but there was a shift towards lower fluorescence intensity in three out of seven healthy donors' and three out of four SLE patients' plasmas analyzed. While not wishing to be bound by theory, and while it is not necessary to practice the present invention, a possible explanation is that, as the cultures of GNPs age, or if there is less available serum, the CNPs accrue greater amounts of hydroxyapatite around each of the particles, allowing less protein to be available to be detected by the 8D10 monoclonal antibody in the flow cytometric technique. Conversely, a calcified exudate sample that was laden with larger crystals when observed under the light microscope, was positive but with a very dim mean fluorescence intensity. This sample was cultured in serum free media, and, after two weeks, it showed peaks of greater intensity of fluorescence in the 8D10 positive region of FL-1, coinciding with the visual appreciation of many motile forms appearing in the culture.

The foregoing is provided for purposes of illustrating, explaining, and describing embodiments of this invention. Modifications and adaptations to these embodiments will be apparent to those skilled in the art and may be made without departing from the scope or spirit of this invention.

We claim:

1. A method of characterizing calcifying nanoparticles (CNPs), comprising:
creating a test sample comprising CNPs isolated from a biological source, a buffer solution, a plurality of calibration beads, and a fluorescent marker specifically linked to the CNPs;
evaluating the test sample using a flow cytometer; and
analyzing results from the flow cytometer to determine a characterizing feature of the calcifying nanoparticles, wherein the characterizing feature is selected from the group consisting of: number of CNPs, concentration of CNPs, size of CNPs, level of CNP aggregation, size and light dispersion characteristics of CNPs, fluorescence intensity of the CNPs when labeled with a specific antibody, and combinations thereof.

2. The method of claim 1, further comprising:
creating an isotype control comprising CNPs isolated from a biological source, the buffer solution, a plurality of calibration beads, and a fluorescent marker that is not linked to the CNPs; and
evaluating the isotype control using the flow cytometer.

3. The method of claim 2, wherein the test solution and the isotype control comprise approximately the same concentration of beads and the beads have a uniform diameter.

4. The method of claim 3, wherein the uniform diameter of the beads is selected so that the beads are larger than the expected size of CNP aggregates in the test sample.

5. The method of claim 3, wherein the heads have a uniform diameter ranging between 5 and 10 micrometers.

6. The method of claim 1, wherein the beads are fluorescent.

7. The method of claim 1, wherein the analyzing step comprises analyzing a plot of side scatter and forward scatter results from the flow cytometer evaluation of the test sample.

8. The method of claim 7, wherein the plot is a log-log plot of side scatter and forward scatter results.

9. The method of claim 1, wherein the selective linkage comprises a monoclonal antibody that specifically binds to the CNPs.

10. The method of claim 9, wherein the selective linkage comprises an antibody that specifically binds to the monoclonal antibody.

11. The method of claim 1, wherein the selective linkage comprises a monoclonal antibody that specifically binds to the CNPs and an antibody that specifically binds to the monoclonal antibody.

12. The method of claim 1, wherein the test sample is produced by creating a first solution comprising CNPs isolated from a biological source, a buffer solution, and a monoclonal antibody that specifically binds to CNPs;
incubating the first sample, wherein the incubating step is of sufficient duration for the monoclonal antibody to hind to the CNPs in the rust sample; and
creating the test sample, by adding a marker to the first sample, wherein the marker comprises a fluorescent molecule and the marker specifically binds to the monoclonal antibody.

13. The method of claim 12, wherein the monoclonal antibody is 8D10.

14. The method of claim 12, wherein the marker comprises an antibody conjugated to a fluorescent molecule, wherein the antibody is produced against the monoclonal antibody.

15. The method of claim 12, wherein the creating step comprises filtering a liquid containing CNPs from the biological source through a 0.2 micron filter, and using a filtrate from the filtering process to create the test sample.

16. The method of claim 2, wherein the test sample is produced by creating a first solution comprising CNPs isolated from a mammalian subject, a buffer solution, and a monoclonal antibody that specifically binds to CNPs;
incubating the first sample, wherein the incubating step is of sufficient duration for the monoclonal antibody to bind to the CNPs in the first sample; and
creating the test sample, by adding a marker to the first sample, wherein the marker comprises a fluorescent molecule and the marker specifically binds to the monoclonal antibody.

17. The method of claim 16, wherein the isotype control is produced by creating a control precursor comprising CNPs isolated from a mammalian subject, a buffer solution, and a monoclonal antibody is non-specific for CNPs;
incubating the control precursor, wherein the incubating step is of approximately the same duration as the incubating step used to produce the test sample; and
creating the isotype control, by adding the marker to the control precursor.

18. The method of claim 17, wherein the monoclonal antibody is 8D10.

19. The method of claim 17, wherein the marker comprises an antibody conjugated to a fluorescent molecule, wherein the antibody is produced against the monoclonal antibody.

20. The method of claim 1, wherein the CNPs isolated from the biological source are obtained from a source selected from the group comprising blood, bodily exudates, abscess fluids, cells, tissue, extracted tissue, and combinations thereof.
